(19) United States
(12) Patent Application Publication
(10) Pub. No.: US 2025/0276055 A1
(43) Pub. Date: Sep. 4, 2025

(54) Visco-elastic Solid Formulation for Oral Delivery of a Biologically Active Agent

(71) Applicant: US BIOLOGIC, INC, Memphis, TN (US)

(72) Inventor: Jolieke Gerdy Van Oosterwijk, Memphis, TN (US)

(73) Assignee: US BIOLOGIC, INC, Memphis, TN (US)

(21) Appl. No.: 19/199,276

(22) Filed: May 5, 2025

Related U.S. Application Data
(63) Continuation-in-part of application No. PCT/US23/36771, filed on Nov. 3, 2023.
(60) Provisional application No. 63/422,956, filed on Nov. 5, 2022.

(51) Int. Cl. A61K 39/145; A61K 9/00; A61K 9/50; A61K 39/00; A61K 39/07; A61P 31/16

(52) U.S. Cl. CPC A61K 39/145; A61K 9/0053; A61K 9/5015; A61K 9/5036; A61K 39/07; A61P 31/16; A61K 2039/545

(57) ABSTRACT

The presently disclosed subject matter relates a formulation for oral delivery of biologically active agent. In one embodiment, single dose formulation for oral delivery of biologically active agent includes a vaccine antigen expression system and a visco-elastic solid carrier configured to microencapsulate the vaccine antigen expression system. The vaccine antigen expression system is a bacterial antigen expression vehicle expressing one or more recombinant viral protein antigens, wherein the bacterial antigen expression vehicle is Bacillus subtilis.

### FIELD OF THE INVENTION

The presently disclosed subject matter relates to visco-elastic solid formulation for oral delivery of biologically active agents such as a vaccine. The presently disclosed subject matter is specifically formulated to overcome challenges inherent to oral vaccine administration. The presently disclosed subject matter further specifically targets mucosal immunity to elicit an effective immune response against one or more specific pathogenic agents.

### BACKGROUND OF THE INVENTION

The first oral vaccine on the market was the oral polio vaccine, which is made of an attenuated live virus that was suspended in a liquid and could be added to a sugar cube to produce a solid with greater stability. The liquid formulation had to be stored frozen and could not be stored for more than 30 days, the sugar cube can be stored for 6 weeks at room temperature (20 C). These formulations are comprised of live attenuated viruses and were not formulated for single dose administration. One sugar cube would hold for example 50-100 doses per cube. The cube would need to be dissolved prior to administration.

Other oral vaccines use sublingual or buccal delivery, which may require retention or resident time in the mouth for up to 60 seconds to effectively dissolve. The use of surfactants like Tween80 in the formulations can present administration difficulties for people and contribute to compliance issues for full vaccination. Also, it is difficult to require patients, especially children to retain something in their mouth for an extended duration. Should the formulation be swallowed too soon, it would render the formulation ineffectual.

Other oral vaccine formulations may also use as hard shelled/shellac type formulations that present a hard shell. This formulation requires the recipient to swallow a pill, which still presents a problem for certain populations.

Another example, specifically to the control of influenza, is the use of red blood cells as an antigenic carrier. However, sourcing of clean red blood cells presents immediate production and scaling challenges, and the sourcing from animals places a limitation in the reduction to practice for this technology for select vegetarian/vegan population.

A need exists in the industry to provide an oral delivery formation that is a single dose, stable at room temperature, provides both mucosal and systemic immunity and is easy for a patient to swallow.

### BRIEF DESCRIPTION OF THE SEVERAL IMAGES OF THE DRAWINGS

The drawings and method of use according to an example form the present invention. The invention description refers to the accompanying drawings.

FIG. 1A shows the presentation of visco-elastic solid formulation units observed over time and temperature as a monitor of physical appearance. The visco-elastic properties of the solid units facilitated a retention in the size and shape of the units assayed.

FIG. 1B shows weight of visco-elastic solid formulation units recorded over time and temperature as a demonstration of shelf-life stability. Visco-elastic solid formulation units were weighed. The weights were variable, but not significantly different throughout the assay.

FIG. 1C shows protein content of visco-elastic solid formulation units recorded over time and temperature as a demonstration of shelf-life stability. The use of a-expressed protein antigen served as a surrogate diagnostic marker for the assay of stability. Visco-elastic solid formulation units were disjoined through reversal of crosslinking to facilitate protein extraction at each time point. The Coomassie shows protein content at week three and 4, with no appreciable difference between the different temperatures.

FIG. 2A shows protein content of visco-elastic solid formulation units measured at different time points during in vitro dissolution as a demonstration of gastric protection and duodenal dissolution. The use of a green fluorescent protein (GFP) expressed protein antigen served as a surrogate diagnostic marker for the in vitro dissolution assay. Visco-elastic solid formulation units were disjoined through reversal of crosslinking to facilitate protein extraction at each time point. The Coomassie shows protein content remains steady throughout the gastric compartment, and dissolves in the duodenum.

FIG. 2B shows protein content of visco-elastic solid formulation units measured at different time points during in vitro dissolution as a demonstration of gastric protection and duodenal dissolution. The use of a H5-expressed protein antigen served as a measure of retention for the invitro dissolution assay. Visco-elastic solid formulation units were disjoined through reversal of crosslinking to facilitate protein extraction at each time point. The western blot shows H5 protein content remains steady throughout the gastric compartment, and dissolves in the duodenum, with subsequent cleavage of the H5 protein.

FIG. 3A. shows induction of antibody creation in a murine model using an ELISA. Serum IgG antibodies as measured in the ELISA show mice develop H5 specific antibodies within two weeks of oral vaccination.

FIG. 3B. shows serum antibody count as measured by ELISA, and serum antibody functionality as measured using hemagglutination inhibition assays, correlate. Upon oral vaccination, the serum IgG antibodies increase, as do the hemagglutination inhibition titers, showing that functional antibodies are produced.

### SUMMARY OF THE INVENTION

This summary describes several embodiments of the presently disclosed subject matter and, in many cases, lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The novel subject matter includes a stable visco-elastic solid formulation for oral delivery of biologically active agent. This formulation is made of a vaccine antigen expression system and a visco-elastic solid carrier configured to encapsulate the vaccine antigen expression system. The visco-elastic solid carrier is formed into a shape for oral consumption. The vaccine antigen expression system is made of a bacterial antigen expression vehicle expressing one or more recombinant viral protein antigens. The vaccine antigen expression system is a bacterial antigen expression vehicle expressing one or more recombinant viral protein antigens, wherein the bacterial antigen expression vehicle is Bacillus subtilis.

In one embodiment, a vaccine antigen expression system encapsulated in a visco-elastic solid product is made by a novel process. The novel process includes the steps of dissolving sodium alginate (2% w/v) and 17% sucrose (w/v) in phosphate buffered saline to form a microencapsulation solution; mixing the vaccine antigen expression system with the microencapsulation solution to form a homogeneous conglomerate mixture comprising the microencapsulate; adding the mixture to a mold having a shape; freezing the mold for a sufficient amount of time and at a sufficient temperature to form a visco-elastic solid microencapsulate formulation.

In another embodiment, a novel method of treating a subject in need thereof is provided. This method functions to stimulate an immune response. In one aspect, the subject in need of treatment to prevent an Influenza infection. This method includes the steps of: providing a formulation for oral delivery of a biologically active agent comprising: a vaccine antigen expression system and a visco-elastic solid carrier configured to microencapsulate the vaccine antigen expression system, wherein the vaccine antigen expression system is comprised of a bacterial antigen expression vehicle expressing one or more recombinant viral protein antigens, vehicle is Bacillus subtilis, and wherein the one or more recombinant viral protein antigens are engineered from hemagglutinin and a visco-elastic solid carrier configured to microencapsulate the vaccine antigen expression system, wherein the visco-elastic solid carrier is formed into a shape for oral consumption.

Results demonstrate that the formulation in the presently disclosed subject matter contributes to the stability of a vaccine antigen expression system and elicits an effective mucosal immune response when administered orally.

### DETAILED DESCRIPTION OF THE INVENTION

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will be controlled. Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment. While the terms used herein are believed to be well understood by one of ordinary skill in the art, definitions are set forth herein to facilitate explanation of the presently disclosed subject matter. Unless defined otherwise, all technical and scientific terms used herein have the same meaning as commonly understood by one of ordinary skill in the art to which the presently disclosed subject matter belongs. Although any methods, devices, and materials similar or equivalent to those described herein can be used in the practice or testing of the presently disclosed subject matter, representative methods, devices, and materials are now described. Following patent law convention, the terms “a,” “an,” and “the” refer to “one or more” when used in this application, including the claims. Thus, for example, reference to “a cell” includes a plurality of such cells, and so forth. All references to singular characteristics or limitations of the present disclosure shall include the corresponding plural characteristic(s) or limitation(s) and vice versa, unless otherwise specified or clearly implied to the contrary by the context in which the reference is made. All combinations of method or process steps as used herein can be performed in any order, unless otherwise specified or clearly implied to the contrary by the context in which the referenced combination is made.

The methods and compositions of the present disclosure, including components thereof, can comprise, consist of, or consist essentially of the essential elements and limitations of the embodiments described herein, as well as any additional or optional components or limitations described herein or otherwise useful.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

The presently disclosed subject matter relates to a composition and method of using the composition for oral delivery of a biologically active agent to a subject. More particularly, the presently disclosed subject matter relates to visco-elastic solid for oral delivery of biologically active agent such as a vaccine.

The term “visco-elastic solid” means the shape may or may not fail depending on the applied stress and the duration thereof, in comparison to a purely elastic material, prolonged stress at a lower force would ultimately cause a break in the polymers. If stress is applied to the delivery unit, the shape will change-viscous, but when the stress is removed, it will ultimately return (“creep”) back to its original shape when the physical stress is removed-elasticity.

The term “microencapsulate” means product generated via the use of the formulation in the presently disclosed subject matter to entrap the individual units of biologically active agent.

The term “microencapsulation” means the product generated via the use of the formulation in the presently disclosed subject matter comprising a blend or amalgam of microencapsulate to entrap the bulk biologically active agents in controlled volumetric units.

The term “bioactive agent,” “biologically active agent,” “bioactive antigenic agent,” “active pharmaceutical agent,” refers to any substance that is of medical or veterinary therapeutic, prophylactic, or diagnostic utility. In some embodiments, the bioactive agent includes a therapeutic agent. As used herein, a therapeutic agent refers to a bioactive agent that, when administered to a patient, will cure, or at least relieve to some extent, one or more symptoms of, a disease or disorder. In some embodiments, bioactive agent includes a prophylactic agent. As used herein, a prophylactic agent refers to a bioactive agent that, when administered to a patient either prevents or ameliorates the occurrence of a disease or disorder or, if administered subsequently to a therapeutic agent, prevents, or retards the recurrence of the disease or disorder. In some embodiments, bioactive agent refers to a bacterial antigen expression vehicle for the expression of antigens that elicit an immune response, or proteins that can modulate the immune system, to enhance therapeutic potential. In some embodiments, the administration of the biologically active antigenic agent can elicit an immune response that is either prophylactic to prevent disease contraction and transmission, or therapeutic to resolve existing disease infection.

In some embodiments, the bioactive agent is a recombinant whole-cell bacteria molecular engineered to express one or more protein antigens. As used herein, the “recombinant whole-cell bacteria engineered to express one or more protein antigens” is a bacterial antigen expression vehicle for the expression of antigens. As used herein, “whole-cell bacteria” refers to bacterial cells, maintained under conditions that retain the bacterial cellular structural integrity, that is, whole-cell structural integrity and antigenicity, as a bioactive recombinant bacterial antigen expression vehicle that is an exogenous protein expression system for the stable presentation of antigen in certain embodiments. Conditions favorable for the structural integrity of the bioactive agent is defined as “stabilized.” In certain embodiments, whole cells will be maintained as stable, not to be broken down into cellular fragments and/or other biological material and/or organelles. In maintaining the stabilized whole-cell structural architecture, some embodiments of vaccine preparation may encompass “active formulations,” defined as live whole-cell bacterial units; other embodiments may encompass “inactive formulations,” defined as killed whole-cell bacterial units termed bacterins. In some embodiments, stabilization refers to the process of bacterial sporulation. As defined and applied herein, sporulation refers to stability through metabolic inactivity.

In some embodiments, the whole-cell bacteria include, but is not limited to preparations of B. subtilis. As used herein, the bioactive agent, or biologically active agent, is a whole-cell bacterial antigen expression vehicle. As used herein cultures of B. subtilis are used as a collective homogeneous, clonally expanded preparation of the viral antigen expression vehicle. As used herein, viral antigen expression vehicles are considered biological vehicles, or biologics, wherein the composition is made of components of living biological organisms. The use of viral antigen expression vehicles as biologics that present prophylactic and/or therapeutic intervention strategies in the control of disease has increased recently given the application of recombinant expression technologies. As a biologic, with added commensalism such as Bacillus subtilis have emerged in the biotechnology space as promising systems for recombinant protein expression technology given their GRAS (Generally Recognized As Safe) determination by the US Food and Drug Administration (FDA). However, B. subtilis strains are only now presented with the expression systems supporting recombinant protein technologies. As such and given that B. subtilis is a commensal microorganism found in the GI tracts of both ruminants and humans, its utility as a viral antigen expression vehicle for orally delivered vaccine administration is shown in the presently disclosed subject matter.

In some embodiments, the bacterial antigen expression system is molecular engineered to express one or more antigens, which are expressed using a recombinant plasmid expression vector transformation event. As used herein, “molecular engineered” refers to the molecular biological technique of biosynthetic molecular cloning of genes identified for the expression of specific proteins of interest into the plasmid expression vector. As used herein, the plasmid expression vector is then used to transform a competent bacteria into the bacterial antigen expression system. In some embodiments, the antigen expression system is vectored to express protein diagnostic tags. As used herein, such proteins are used in the processes of assaying and evaluating the functionality of the expression system in the context of a targeted administration subject. In some embodiments, the recombinant bacteria are engineered to express fluorescent proteins, examples herein include, but are not limited to GFP, iRFP720.

In some embodiments, the bacterial antigen expression system is a viral vectored for the expression of biological antigens of viral origin. In some embodiments, a viral-vectored bacterial antigen expression system is engineered to provide a vaccine for oral vaccination. The term “viral-vectored bacterial antigen expression system” as used herein refers to the recombinant expression protein antigens derived from viral infectious agents of disease vectored in the context of the whole-cell bacterial antigen expression system for vaccination.

In some embodiments, the recombinant expression systems are molecular engineered to express immunomodulating agents. As used herein, immunomodulating agents refer to protein antigens that elicit a specific immunological response to the antigen in the form of serological antibody production. Examples of immunomodulating agents include but are not limited to adjuvants. As used herein, an adjuvant is a substance that augments the immunological response to the vaccine antigen.

A composition and method are needed for the stable presentation of antigen, in the context of a whole-cell bacterial antigen expression vehicle and administered in the context of an encapsulate, as an orally administered vaccine for the control of infectious disease.

In some embodiments, the microencapsulate is in an amount of about 5% to about 99% w/w of the composition. In some embodiments, the microencapsulate is in an amount of about 1% to about 95% w/w of the composition. In some embodiments, the effective amount of the bioactive agent is an immunogenically effective amount with the minimal immunizing dosage (MID) of about 5×10³ to about 5×10¹³ sporulated bacteria per 1 mL unit. In some embodiments, cross-linking agent is in an amount of about 0.5% to about 15% w/w of the composition.

In some embodiments, the visco-elastic encapsulate is presented in a volumetric range of 1 mL to 20 mL. In some embodiments, the visco-elastic encapsulate is presented as a shape, including but not limited to a bone, dinosaur, heart, fruit, or animal. In some embodiments, the visco-elastic encapsulate is colored using food coloring. In one embodiment the formulation provides a single dose but can also be configured to provide multiple doses.

In one embodiment, the shape is a linear extrusion that can be formed into a roll. The roll can include perorations to delineate a dosage. Multiple dosages can be included in a stable shelf roll.

Further, regardless of the particular mode and timing of administration used in accordance with the methods of the presently disclosed subject matter, the bacterium, including the bacterium-based compositions and antigenic agents described herein, are typically administered in an amount effective to achieve the desired response (i.e., protection against the infectious agents). As such, the term “effective amount” is used herein to refer to an amount of the therapeutic composition sufficient to produce a measurable biological response (e.g., an immune response against an infection). In this regard, in some embodiments, the term “therapeutically effective” is used interchangeably herein with the phrase “immunogenically effective” to refer to an amount of whole-cell bacterial antigen expression vehicle expressing the at least one or more antigenic agents of the presently disclosed subject matter sufficient to induce an effective immune response in a host against an infectious agent. Actual dosage levels of active ingredients in a therapeutic composition of the presently disclosed subject matter (can be varied so as to administer an amount of a composition that is effective to achieve the desired therapeutic response for a particular subject and/or application. Of course, the selected dosage level and amount of the bacterium and the other components of such a composition will depend upon a variety of factors including the activity of the bacterium, formulation, the route of administration, combination with other drugs or treatments, severity of the condition being treated, and the physical condition and prior medical history of the subject being treated. Preferably, a minimal dose is administered, and dose is escalated in the absence of dose-limiting toxicity to a minimally effective amount. Determination and adjustment of a therapeutically effective dose, as well as evaluation of when and how to make such adjustments, are known to those of ordinary skill in the art. As used herein, for example, in some embodiments, the therapeutic effective dose of the at least one antigenic agent is of a minimum immunizing dosage (MID) as measured in active antigen-expressing sporulated bacteria of about 5×10³ to about 5×10¹³ CFU. The presently disclosed subject matter, in some embodiments, provides a method of controlling infectious diseases by vaccinating a subject at risk for being a carrier and/or symptomatic host thereof. The method includes orally administering to the subject a composition as disclosed herein. In some embodiments, the presently disclosed subject matter relates to a method of orally administered biological-based vaccines utilizing antigen-expression systems made of vectored bacteria that, upon consumption, elicit an immune response in targeted populations.

### Example 1 Visco-Elastic Solid Encapsulate Preparation

Sodium alginate (2% w/v) was dissolved in phosphate buffered saline, with 17% Sucrose (w/v) as a stabilizer, and 1% fermented sugar (w/v) as an antimycotic. In some formulations, Xanthan Gum (w/v) was used as a thickening agent. In some formulations, Citric Acid is used as a natural preservative. As provided in Table 1, the percentage mix of the various components in the contribution to the overall stability of the formulation were determined empirically and remain unique to the stability of the visco-elastic solid formulation units.

This solution is used for encapsulation of the vaccine antigen expression system (the hemagglutinin (HA) expressed by B. subtilis) as described in Example 4. Briefly, the hemagglutinin (HA) expressed by B. subtilis vaccine is mixed to homogeneity. Encapsulate solutions containing the vaccine, is poured into molds. Molds are placed in the −80 C freezer for a minimum of 1 hr, and maximum of 18 hrs. Frozen shapes are then placed into a crosslinking solution which can be either 1) 6% Calcium Chloride (w/v), 2) 2% Calcium Lactate (w/v), or 3) 2% Calcium Carbonate (w/v), and put on a stir plate for constant and ubiquitous crosslinking by intercalation.

TABLE 1

| For- | Alginate | Fermented | Xanthan | Citric | Calcium | Calcium |
|---|---|---|---|---|---|---|
| mulation | % | Sugar | Gum | Acid | formulation | % |
| 1 | 2 | — | — | Y | Lactate | 2 |
| 2 | 2 | Y | — | Y | Lactate | 2 |
| 3 | 2 | — | Y | Y | Lactate | 2 |
| 4 | 2 | Y | Y | Y | Lactate | 2 |
| 5 | 2 | — | — | — | Chloride | 6 |
| 6 | 2 | Y | — | — | Chloride | 6 |
| 7 | 2 | — | Y | — | Chloride | 6 |
| 8 | 2 | — | — | Y | Chloride | 6 |
| 9 | 2 | Y | Y | — | Chloride | 6 |
| 10 | 2 | — | Y | Y | Chloride | 6 |
| 11 | 2 | Y | — | Y | Chloride | 6 |
| 12 | 2 | Y | Y | Y | Chloride | 6 |
| 13 | 2 | — | — | Y | Carbonate | 2 |
| 14 | 2 | — | Y | Y | Carbonate | 2 |
| 15 | 2 | Y | — | Y | Carbonate | 2 |
| 16 | 2 | Y | Y | Y | Carbonate | 2 |

### Example 2 Stability

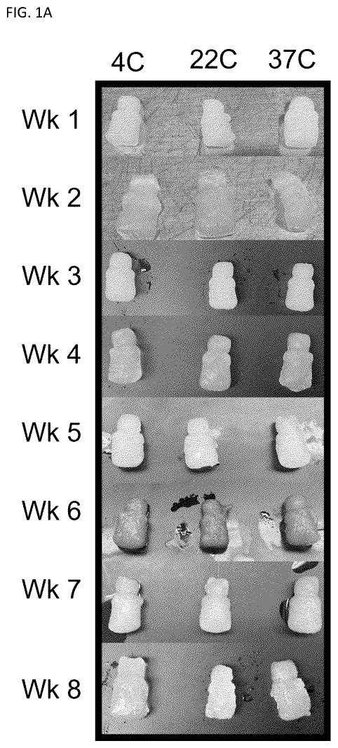

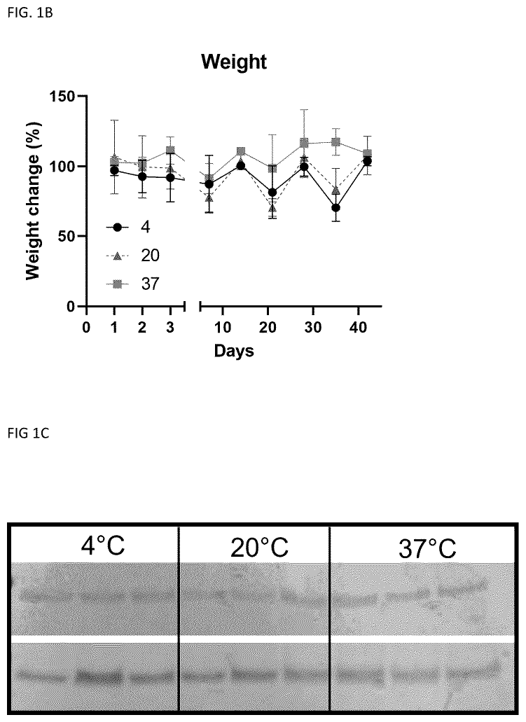

Now referring to FIGS. 1A-C, visco-elastic solid formulation units were created and incubated at either 4 C, 22 C (RT), or 37 C. The visco-elastic solid formulation units were placed in a 6 well plate (3 per well, for sampling at different time points). The visco-elastic solid formulation units were kept at the respective temperatures for 8 weeks and sampled every day for the first three days, and then every week. FIG. 1A shows the time points and sampling sizes, a total of six gummies were sampled prior to start, and then three visco-elastic solid formulation units for each time point from each temperature. To assess the physical stability of the visco-elastic solid formulation units, FIG. 1B demonstrates that the overall weight of the units remained constant as a function of time and temperature. To assess the encapsulation stability of the visco-elastic solid formulation units, FIG. 1C demonstrates that the overall expression of a surrogate protein antigen expression system remained relatively stable and constant as a function of both time and temperature. Visco-elastic solids were formulated with B. subtilis spores engineered to express the fluorescent protein GFP. Whole protein was extracted from visco-elastic solid formulation units over time at various assay temperatures and resolved via SDS-PAGE. Gels were stained via Coomassie.

### Example 3 Dissolution Assays

The in vitro dissolution assays were modeled after USP guidelines, combined with consulting the scientific literature. Martinez, Marilyn N. et al. “Veterinary Application of In Vitro Dissolution Data and the Biopharmaceutics Classification System.” (2013) USPC; Corstens, Meinou N. et al. “Emulsion encapsulation in calcium-alginate beads delays lipolysis during dynamic in vitro digestion.” (2018) J of Functional Foods.

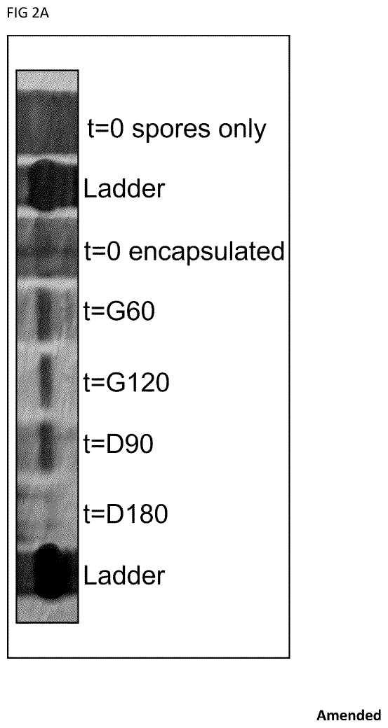

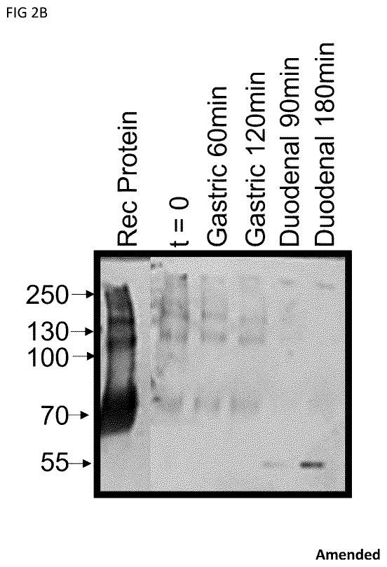

Now referring to FIGS. 2A and 2B, the stability of visco-elastic solid formulation units throughout the gastro-intestinal tract was evaluated as a function of protein antigen expression. Visco-elastic solids were formulated with B. subtilis spores engineered to express either the fluorescent protein GFP (2A) or the influenza antigen H5 (2B). The majority of visco-elastic solid formulation units presented a drop in weight after one hour in stomach solution (pH<2.0) of the dissolution assay, indicating dehydration. At the duodenal stage, the visco-elastic solid formulation units expanded, while the weight remained constant, suggesting that the units are rehydrating and dissolving (pH˜7.0). The silver stain in FIG. 2A shows the GFP concentration in the visco-elastic solid formulation taken at different time points (starting from prior to the assay start (t=0), at one and 2 hours in the gastric compartment (G60 & G120), and 1.5 and 3 hours in the duodenal compartment (D90 & D180)). The western blot in FIG. 2B was probed with commercial anti-H5 antigen (Sinobiological) and shows purified recombinant protein (Rec Protein) versus H5 protein recovered from visco-elastic solid formulations through gastric and duodenal compartments. The clear decrease in protein concentration in the visco-elastic solid formulation units indicates protein release from the visco-elastic solid formulation units. The cleavage of the H5 protein by enzymes in the duodenal compartment demonstrate functionality of the protein released in the duodenal compartment.

### Example 4 In Vivo Data

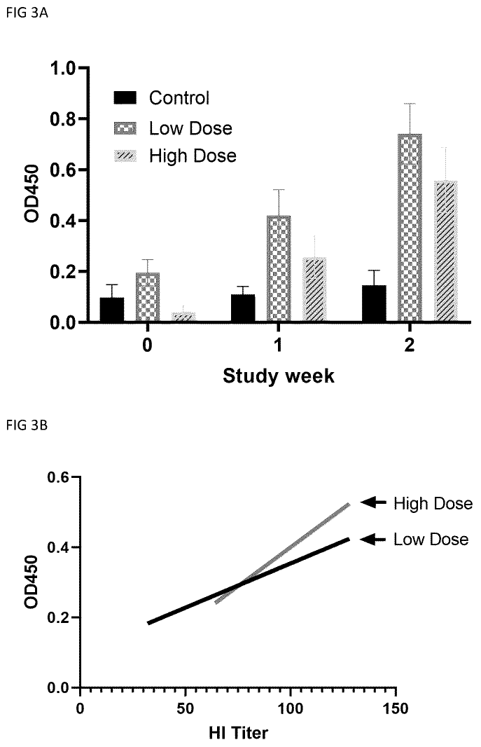

CD-1 mice were dosed with visco-elastic solid formulation units containing either empty vectored (Control) or H5-vectored B. subtilis spores, on day 0. Vaccine formulations were either of low dose (10E8) or high dose (10E10). Mice fed ad libitum on the visco-elastic solid formulation units. Blood was collected using submandibular vein bleed on a weekly basis, with the first bleed before exposure to antigen. Serum IgG antibodies specific for H5 were evaluated using ELISA. Now referring to FIG. 3A, serum HA-specific IgG antibodies increased in the first two weeks post dosing.

To evaluate the functionality of antibodies produced by dosing with the visco-elastic solid formulation units, hemagglutination inhibition (HI) assays were performed. HI assays are standard of practice in influenza vaccine testing (Kaufman et al 2017 (An Optimized Hemagglutination Inhibition (HI) Assay to Quantify Influenza-specific Antibody Titers (nih.gov)). Now referring to FIG. 3B, HI titers were found to correlate with ELISA detection of H5 serum antibodies. These results are demonstrating immunogenicity of the oral vaccine when administered via the presently disclosed subject matter: visco-elastic solid formulation units.

Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment. While the terms used herein are believed to be well understood by one of ordinary skill in the art, definitions are set forth herein to facilitate explanation of the presently disclosed subject matter. While the invention has been described with reference to details of the illustrated embodiments, these details are not intended to limit the scope of the invention as defined in the appended claims. The embodiment of the invention in which exclusive property or privilege is claimed is defined as follows.

We claim:

1. A stable visco-elastic solid formulation for oral delivery of biologically active agent comprising: a vaccine antigen expression system and a visco-elastic solid carrier configured to encapsulate the vaccine antigen expression system, wherein the visco-elastic solid carrier is formed into a shape for oral consumption, wherein the vaccine antigen expression system is comprised of a bacterial antigen expression vehicle expressing one or more recombinant viral protein antigens.

2. The formulation of claim 1, wherein the vaccine antigen expression system is comprised of a bacterial expression vehicle expressing one or more recombinant fluorescent proteins as a diagnostic marker.

3. The formulation of claim 1, wherein the one or more recombinant viral protein antigens are engineered from influenza.

4. The formulation of claim 2, wherein the one or more recombinant fluorescent protein markers are used in the differentiation of infected and vaccinated subjects.

5. The formulation of claim 1, wherein the bacterial antigen expression vehicle is Bacillus subtilis.

6. The formulation of claim 1, wherein the visco-elastic solid formulation is stable at room temperature for at least 8 weeks.

7. The formulation of claim 1, wherein the shape is configured to provide a plurality of doses.

8. The formulation of claim 1, wherein the visco-elastic solid carrier has a volumetric range of between 1 mL to 20 mL.

9. The formulation of claim 1, wherein visco-elastic solid carrier is molded into a shape selected from the group consisting of: a bone, a dinosaur, a heart, a fruit, and an animal shape.

10. A stable visco-elastic solid product configured for oral delivery of biologically active agent made by the process comprising:
dissolving sodium alginate (2% w/v) and sucrose (w/v) in phosphate buffered saline to form an encapsulation solution;
mixing an antigen expression system with the encapsulation solution to form a mixture;
adding the mixture to a mold having a shape; and
freezing the mold for a sufficient amount of time and at a sufficient temperature to form a visco-elastic solid product.

11. The product of claim 10, wherein the sufficient temperature to form a visco-elastic solid product is −80 C.

12. The product of claim 10, wherein the sufficient amount of time is between 1 hour to 18 hours.

13. The product of claim 10, wherein the visco-elastic solid product is stable at room temperature for at least 8 weeks.

14. The product of claim 10, wherein the visco-elastic solid product has a volumetric range of between 1 mL to 20 mL.

15. The product of claim 10, wherein the shape is configured to provide a plurality of doses.

16. A method of treating a subject in need thereof comprising:
providing a stable solid formulation for oral delivery of biologically active agent comprising: a vaccine antigen expression system and a visco-elastic solid carrier configured to encapsulate the vaccine antigen expression system, wherein the vaccine antigen expression system is comprised of a bacterial antigen expression vehicle expressing one or more recombinant viral protein antigens, vehicle is Bacillus subtilis, and wherein the one or more recombinant viral protein antigens are engineered from hemagglutinin and a stable visco-elastic solid carrier configured to encapsulate the vaccine antigen expression system, to stimulate an immune response, wherein the visco-elastic solid carrier is formed into a shape for oral consumption.

17. The method of claim 16 wherein the subject is in need of treatment to prevent an Influenza infection.

18. The method of claim 16 wherein the shape is configured to provide a plurality of doses.

19. The method of claim 16 wherein the shape is configured to provide a single dose.
